(19) United States
(12) Patent Application Publication
(10) Pub. No.: US 2014/0107605 A1
(43) Pub. Date: Apr. 17, 2014

(54) Combined Leg Cuff and Leg Gather

(71) Applicants: Charles F. Schroer, JR., Duluth, GA (US); Steven D. Linton, Duluth, GA (US)

(72) Inventors: Charles F. Schroer, JR., Duluth, GA (US); Steven D. Linton, Duluth, GA (US)

(21) Appl. No.: 14/054,244

(22) Filed: Oct. 15, 2013

Related U.S. Application Data
(60) Provisional application No. 61/713,721, filed on Oct. 15, 2012.

(51) Int. Cl. A61F 13/494

(52) U.S. Cl. CPC A61F 13/49413
USPC 604/385.22; 604/385.23

(57) ABSTRACT

A disposable absorbent article, such as a diaper having a pair of waist sections, a crotch section disposed between the waist sections, a liquid pervious topsheet, a liquid impervious backsheet, an absorbent core disposed between the topsheet and the backsheet, a leg cuff attached to the topsheet at least in part parallel to a longitudinal axis at a first attachment region, a leg gather section which runs along an outer edge region of the crotch section, wherein the leg gather section comprises an outer boundary and an inner boundary, wherein the outer boundary is defined by the edge of the crotch region and the inner boundary is defined by the first leg cuff attachment region; and wherein the leg gather region and leg cuff are attached together at least in part at a second attachment region.

### CROSS-REFERENCE(S) TO RELATED APPLICATION(S)

This application claims priority to U.S. Provisional Patent Application No. 61/713,721, filed Oct. 15, 2012, the entire contents of which is expressly incorporated by reference.

### TECHNICAL FIELD

Embodiments of the present application relate generally to disposable absorbent garments and, more particularly, to a disposable absorbent garment having an upstanding leg cuff and leg gather attached together.

### BACKGROUND OF THE INVENTION

This section is intended to introduce various aspects of the art, which may be associated with exemplary embodiments of the present invention. This discussion is believed to assist in providing a framework to facilitate a better understanding of particular aspects of the present invention. Accordingly, it should be understood that this section should be read in this light, and not necessarily as any admission of prior art.

Infants, toddlers, and incontinent persons wear disposable garments to receive and to contain discharge, such as feces and urine. Disposable garments function both to contain the discharge and to isolate these materials from the body of the wearer and from the wearer's surroundings.

Disposable absorbent garments may include disposable diapers, disposable pull-on garments, adult incontinent articles, and the like. These garments are worn about the lower torso or waist of the user so as to receive and contain urine and other bodily wastes. The principal elements of a typical disposable absorbent garment include a liquid permeable inner layer that is closest to the wearer (or topsheet), a liquid impermeable outer layer (or backsheet) and an absorbent core, sandwiched between the inner and outer layers. The topsheet functions to wick moisture away from the wearer. The absorbent core absorbs and stores the urine and other waste. The backsheet functions to protect the exterior environment from urine and waste. These layers may be combined with other materials, layers, or features to specifically adapt the disposable absorbent garment to a particular purpose or to enhance the activity of the disposable absorbent garment. Elastic members may be incorporated into a part or area of the garment to provide correct fitting or sealing of the garment to the user, such as in the waist.

One of the biggest problems with disposable diapers is designing them so as to comfortably minimize leakage into the surrounding environment. Many designs have been developed to satisfy this need, such as a double leg barrier configuration which uses both an inner elastic upstanding leg cuff and an outer elastic gathering. This double leg barrier configuration was developed in order to enhance leakage protection. The upstanding leg cuff acts to stop the flow of liquid, while the leg gather provides an excellent fit around the wearer's leg and crotch. Examples of double barrier cuffs are seen in U.S. Pat. No. 4,695,278 to Lawson, and U.S. Pat. No. 4,704,116 to Enloe.

As the usage of disposable absorbent garments has expanded, their complexity has increased with the incorporation of additional features serving to enhance their performance and appearance. As a result, the costs of materials and the complexity of manufacturing processes have increased in conjunction with this increase in complexity. For example, the double leg barrier configuration includes at least two elastic strands in the outer leg gather and at least two elastic strands in the inner leg cuff. Hence, there is a need to continue to provide enhanced leakage protection, while reducing the costs historically associated with the double leg barrier construction.

### BRIEF SUMMARY OF THE INVENTION

It is, therefore, an object of certain embodiments of the present invention to provide a leg cuff construction that provides desirable leakage protection. Another object of certain embodiments of the present invention is to provide a leg cuff construction that employs reduced amounts of materials. Still another object of certain embodiments of the present invention is to re-direct elastic forces to improve fit around the legs and hips.

According to one aspect of the present invention, a general embodiment of a disposable absorbent garment is described. The disposable absorbent garment comprises a pair of waist sections, a crotch section disposed between the waist sections, a liquid pervious topsheet, a liquid impervious backsheet, an absorbent core disposed between the topsheet and the backsheet, a leg cuff attached to the topsheet at least in part parallel to a longitudinal axis at a first attachment region, a leg gather section which runs along an outer edge region of the crotch section, wherein the leg gather section comprises an outer boundary and an inner boundary, wherein the outer boundary is defined by the edge of the crotch region and the inner boundary is defined by the first attachment region, and wherein the leg gather region and leg cuff are attached together at least in part at a second attachment region.

In embodiments disclosed herein, the leg gather section comprises no elastic strands, one elastic strand, two elastic strands, or three elastic strands. The leg cuff may also comprise no elastic strands, one elastic strand, two elastic strands, or three elastic strands. In another embodiment, the leg gather section and/or the leg cuff include more than three elastic strands. Various combinations of the number of elastic strands within the leg cuff and leg gather may exist, such as two elastic strands in the outer leg gather and none in the inner leg cuff. As an additional example, the leg cuff may comprise one elastic strand while the leg gather comprises none, the leg cuff may comprise no elastic strands while the leg gather comprises one, or both the leg cuff and leg gather may comprise one elastic strand. Additionally, the second attachment region may be continuous along a line at least in part parallel to the longitudinal axis, or within a 30°-60° angle to the longitudinal axis or the second attachment may comprise one or more individual point attachments. In an embodiment of the disclosure, the elastic strands are at least in part, parallel to the lines of angular force within the diaper and perpendicular to the lines of angular force created by the waist and elastic side panels. In another embodiment, the combined leg cuff provides additional angular forces that can be adjusted and/or optimized for a desired fit around the wearer's leg. In one embodiment, the combined leg cuff, running in a longitudinal direction, is disposed between a first angular force and second angular force. In one embodiment, the first angular force is disposed between the combined leg cuff and the back waist region, and the second angular force is disposed between the combined leg cuff and the front waist region. In yet another embodiment, the first angular force has an angle of between about 120-170 degrees, preferably about 150 degrees with respect to the direction of the combined leg cuff and the back waist region. In another embodiment, the second angular force has an angle of between about 40-80 degrees, preferably about 60 degrees, with respect to the direction of the combined leg cuff. In yet another embodiment, the absorbent article includes a third angular force and a fourth angular force disposed near the back waist region, the third and fourth angular forces preferably have angles between 30-70 degrees, preferably 50 degrees, with respect to the force direction in the side panel region.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing.

FIG. 1 is an extended view of the inside of an example diaper. The right side of the diaper shows an embodiment where there are three elastics in the outer leg gather and none in the leg cuff. The left side of the diaper shows an embodiment where there are two elastic strands within the leg cuff and none in the leg gather.

FIG. 2 is a closer view of the right side of the diaper of FIG. 1.

FIG. 3 is a closer view of the left side of the diaper of FIG. 1.

FIG. 4a is a cross section through the crotch section of a conventional diaper. FIG. 4b is a cross section through the crotch section of the diaper of FIG. 1 further illustrating two embodiments of the invention on the left and right sides.

FIG. 5 illustrates different angular forces during the use of a diaper.

FIG. 6 illustrates an embodiment of the invention where one or more elastics are attached at an angle from the side panel to the inner leg cuff anchor, adjacent the absorbent core.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. Also, for simplification purposes, there may be only one exemplary instance, rather than all, is labeled. It should be understood, of course, that this disclosure is not limited to the particular embodiments illustrated herein.

### DETAILED DESCRIPTION OF THE INVENTION

As described previously, various aspects of embodiments of the present invention are particularly suited to or for a disposable absorbent garment, such as baby diapers or training pants. To illustrate the invention and preferred embodiments of the invention, much of the following Detailed Description will be provided in the context of such disposable absorbent garments. It is contemplated that various aspects of the inventive composite, garment, system and process may be applicable to other material structures and processes. This Detailed Description and exemplary embodiments should not be construed, therefore, as limiting the invention to the structures, configurations, methods, and processes described herein.

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or article described herein can be implemented with respect to any other method or article described herein.

As used herein, “attached” means that the parts are integral with each other or are separate components that have been connected to each other. As used herein, a leg cuff attached region refers to area in which the leg cuff is directly attached to the top sheet, or it refers to the exterior standing edge of the leg cuff, if the leg cuff is integral to the top sheet.

As used herein, “disposable absorbent garment” or “disposable absorbent article” refers to a disposable garment which absorbs and contains body exudates. Typically, the articles are intended to be discarded after a limited period of use. Exemplary embodiments of disposable absorbent articles are diapers, training pants, youth pants, and adult incontinent products.

As used herein, “absorbent core” refers to the materials of the absorbent garment that are intended to provide the primary absorbency capabilities of the article.

As used herein, the term “longitudinal axis” refers to the imaginary line running through the center of the diaper bisecting the back waist region, the crotch region, and the front waist region.

As used herein, the term “chassis” is used to refer to the region that runs along the longitudinal axis of the diaper and includes the absorbent core.

As used herein, “inner” or “inside” means closer to the longitudinal axis. As used herein, “outer” or “outside” means farther from the longitudinal axis.

As used herein, the term “inner leg cuff” or “leg cuff” refers to a cuff which runs, at least in part, parallel to the longitudinal axis and is located inside of the outer leg gather. The inner leg cuff is attached to the topsheet of the garment at a first attachment region.

As used herein, the term “outer leg gather” or “leg gather” refers to the outer edge region of the crotch portion of the diaper and runs, at least in part, parallel to the longitudinal axis. The outer leg gather section comprises an outer boundary and an inner boundary. The outer boundary is defined by the outer edge of the crotch region and the inner boundary is defined by the inner leg cuff attachment region.

As used herein, the term “combined leg cuff” refers to an inner leg cuff that is attached to an outer leg gather at an additional attachment point. In one example of a combined leg cuff, the outer leg gather and the inner leg cuff share elastic capabilities.

As used herein, “wearer” or “user” refers to the individual that will wear or is wearing the disposable absorbent garment. Exemplary embodiments of wearers are babies and toddlers, minors and adults. “Care taker” refers to an individual that puts on and/or removes the garment from the wearer. The care taker may also be a wearer.

As used herein, the term “about” refers to calculations that are within the standard error of the measuring tool.

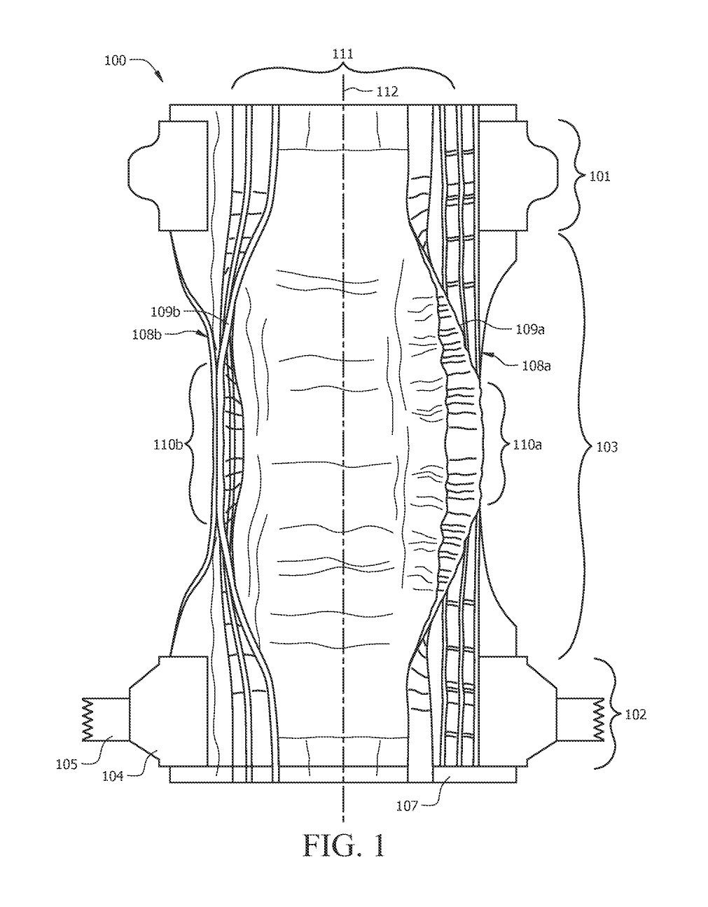

FIG. 1 illustrates a disposable absorbent garment 100 in accordance with an embodiment of the present application. The disposable absorbent garment 100 is of a type that can be placed against or in proximity to the body of a wearer to absorb and contain various bodily exudates. The embodiment of FIG. 1 is introduced to illustrate some basic features of a disposable diaper, most of which are also applicable to other disposable absorbent garments contemplated by the present application. Garment 100 is shown in the unfastened configuration, with the elastic stretched out. Disposable garment 100 includes three main regions aligned along an imaginary longitudinal axis 112. These regions include a first waist region 101, typically located at the front of the user when the garment is worn, a back waist region 102, and a crotch region 103. The shape of garment 100 may vary, and includes a generally rectangular shape, a “T” style shape, an “I” style shape, an hourglass shape, a combination of these shapes, or the like.

Disposable garment 100 waist regions may include ear regions or side panels 104 that extend generally perpendicular to the longitudinal axis at waist regions 101 and 102. In this way, the front 101 and back 102 waist regions may be brought together and attached at the ear region to form openings to accommodate the wearer's legs. The shapes and configurations of ear portions 104 may be formed in any manner to accomplish the particular purposes of disposable garment 100. For example, ear portions 104 may be configured to be permanently or refastenably attached. Additionally, ear portions 104 may be known as side panels.

Disposable garment 100 also comprises fastener means 105 to attach the front 101 and back 102 waist portions of the garment together, to form an opening for the leg of the wearer. These fasteners 105 may be integrated onto the backsheet or topsheet, or may be attached to the topsheet or backsheet (not shown) as additional tabs. Examples of fasteners 105 may include hook and loop fasteners, buttons, pins, snaps, adhesive tape fasteners, cohesives, mushroom and loop fasteners, or the like.

More specifically, loop type fasteners typically comprise a fabric or material having a base or backing structure and a plurality of loop members extending upwardly from at least one surface of the backing structure. The loop material can be formed of any suitable material, such as acrylic, nylon or polyester, and can be formed by methods such as warp knitting, stitch bonding or needle punching. Suitable loop materials are known in the art and are available, for example, from manufacturers such as 3M, St. Paul Minn. (designated as a KN series loop). The loop material can be an integral portion of the outer surface of the disposable garment 100 rather than a separated material or structure.

Hook type fasteners typically comprise a fabric or material having a base or backing structure and a plurality of hook members extending upwardly from at least one surface of the backing structure. In contrast to the loop type fasteners, which desirably comprise a flexible fabric, the hook material advantageously comprises a resilient material to minimize unintentional disengagement of the fastener components as a result of the hook material becoming deformed and catching on clothing or other items. The term “resilient” as used herein refers to an interlocking material having a predetermined shape and the property of the interlocking material to resume the predetermined shape after being engaged and disengaged from a mating, complementary interlocking material. Suitable hook material can be molded or extruded of nylon, polypropylene or another suitable material. Suitable single-sided hook materials for fasteners 105 are known in the art and are available, for example, from manufacturers such as 3M, St. Paul Minn. (designated as a CPL series loop).

Fasteners 105 are located at the back 102 waist region of garment 100. For example, in the representative embodiment of FIG. 1, fasteners 105 are shown on the back waist region 102 and are attached to ear portions 104 located on the back waist region 102 of disposable garment 100. In a preferred embodiment, fasteners 105 are configured to engage with and fasten to an outer surface of front waist region 101 when absorbent article 100 is worn. Referring to FIG. 1, in a preferred embodiment, front waist region 101 further comprises front waist ear portions 113 shaped to provide a grasping area for the convenience of the care taker when putting absorbent article 100 on a wearer. In certain embodiments, fasteners 105 comprise a hook member while a corresponding loop member is disposed on an outer surface of front waist region 101.

Additionally, absorbent garment 100 may comprise elastic portions to help conform the absorbent diaper to the wearer's body. For example, generally some part of the back waist region 102 comprises elastic material 107 to fit the garment to the wearer's waist. The back waist region, the front waist region, or both the front and back waist region may comprise elastic materials. Also, both lateral sides of the crotch region 103, also known as the leg gathers 108, may comprise elastic material 200. Leg cuffs 109, which are attached to either side of the absorbent core, may also comprise elastic material 200. Suitable material for use as elastic members 200 are well known to those skilled in the art. An embodiment of elastic material 200 construction is to attach sheets or strands of ribbons of polymeric, elastomeric material and adhere them to a sheet in the extended configuration. These elastic members 200 may include polyurethane, synthetic rubber, natural rubber, or any combination thereof. Exemplary embodiments of elastic members 200 that may be used with the present invention are given in U.S. Pat. No. 4,892,536 issued Jan. 9, 1990 to DesMarais et al., U.S. Pat. No. 5,540,796 issued Jul. 30, 1996 to Fries, U.S. Pat. No. 4,895,568 issued Jan. 23, 1990 to Enloe, and U.S. Pat. No. 5,196,000 issued Mar. 23, 1993 to Clear et al.

As mentioned above, the diaper includes leg cuffs 109 that run along the longitudinal axis close to the leg edge of the diaper next to the outer leg gather 108. These leg cuffs 109 provide extra sealing to keep excess fluids and material inside of the diaper. The leg cuffs 109 may have additional elastic material in them to better fit the wearer, and may also comprise absorbent material to prevent leakage. Exemplary embodiments of cuffs are given in U.S. Pat. No. 4,704,116 issued Nov. 3, 1987 to Enloe, U.S. Pat. No. 5,582,606 issued Dec. 10, 1996 to Bruemmer et al., U.S. Pat. No. 4,795,454 issued Jan. 3, 1989 to Dragoo, and U.S. Pat. No. 5,085,654 issued Feb. 4, 1992 to Buell.

According to the present disclosure, in order to reduce the amount of material, and thereby the cost of the diaper, the leg cuff 109 and leg gather 108 of the diaper are attached together in the crotch region 103 of the diaper 100. In this way, the leg cuff 109 and leg gather 108 are able to share elastic materials 200, which reduces the amount of elastic material used in the diaper, while still maintaining enhanced leakage protection. This attachment is in addition to the first attachment of the leg cuff to the topsheet. For example, the leg cuff 109 and leg gather 108 are combined together using adhesive, sonic bonding, or mechanical bonding. The leg cuff 109 and leg gather 108 are attached together at specific points, or they may also be attached continuously. The additional attachment area may be skewed towards the back the garment 103, skewed towards the front of the garment 102, or directly in the middle. The length of the additional attachment may also vary. For example, the additional attachment may comprise only one point in or near the middle of the crotch, or the attachment may span 1 inch, 2 inches, 3 inches, and so forth. It is understood that the numbers provided are exemplary as it is understood that the attachment may span as much as the distance between the side panels 104 and the front ears 113.

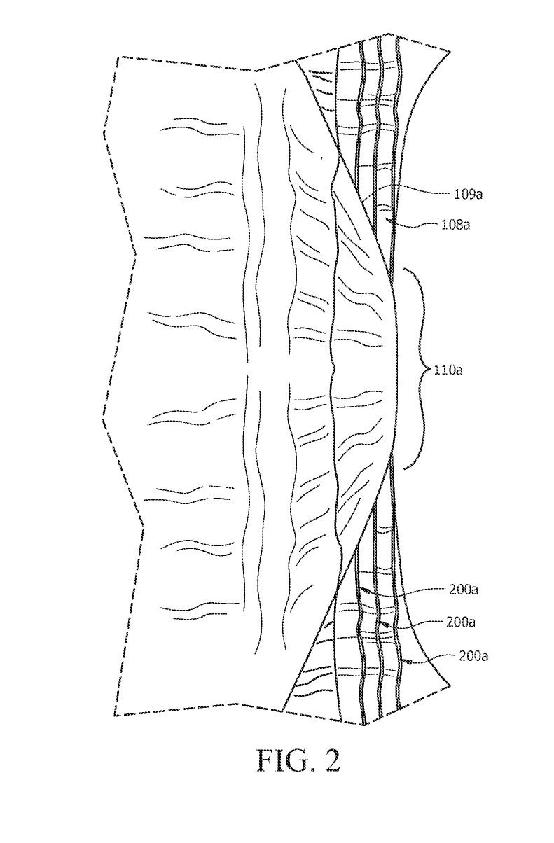

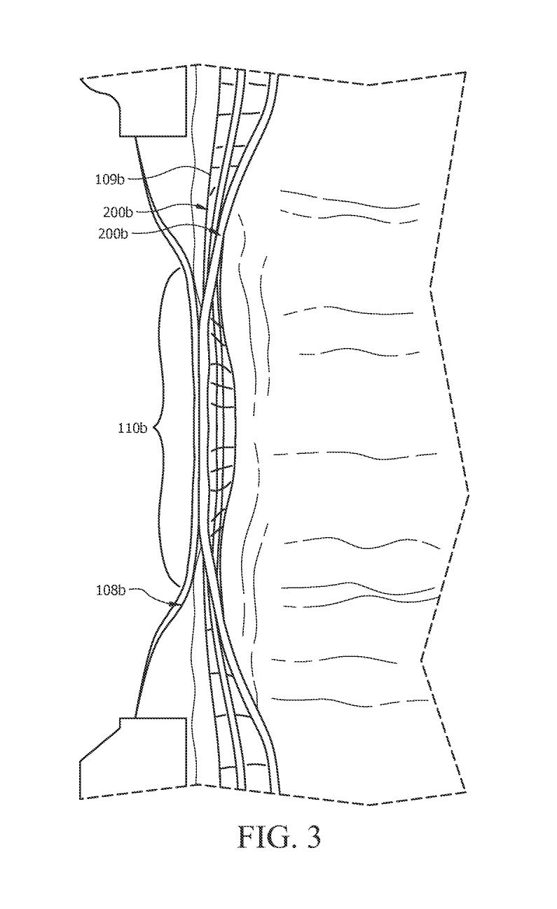

Current diapers tend to have three elastic strands 200 in the outer leg gather 108 and two in the inner leg cuff 109; however, some diapers may contain more or less. In contrast, and according to embodiments of the disclosure, some of the historical elastic strands 200 may be removed from the construction of the diaper. For example, referring to FIGS. 1 and 3, in the left crotch side of the diaper pictured in FIG. 1 the leg gather 108b does not comprise any elastic, while the leg cuff 109b comprises two elastic strands 200b. The inner leg cuff 109b and outer leg gather 108b are attached together along a length which creates a combined leg cuff 110b. Attachment of the leg gather 108 to the elasticized leg cuff 109 allows the leg gather and the leg cuff to share the elastication. Referring to FIGS. 1-2, in the right crotch side of the diaper pictured in FIG. 1, the leg gather 108a comprises three elastic strands 200a, while the leg cuff does not comprise any elastic strands. The leg cuff 109a and leg gather 108a on the right side are attached together along a length which creates a combined leg cuff 110a. It should be understood that these drawings illustrate two different embodiments of the invention which can be implemented separately. That is, while the right and left sides of FIG. 1 are different, diapers using the combined duel cuff may use the same configurations of elastic on both the left and right side, rather than the different configurations shown in FIG. 1.

In addition to varying the number of elastic strands 200, the configurations of elastic strands 200 within the combined leg cuff may vary. That is, either the outer leg gather 108 or the inner leg cuff 109 may comprise elastic, while the other comprises none. Additionally, both the inner leg cuff 109 and outer leg gather 108 may comprise elastic strands 200. In one example the outer leg gather 108 comprises 2 elastic strands 200, while the inner leg cuff 109 comprises 1 elastic strand 200. In this example, the combined leg cuff comprises a total of 3 elastic strands 200. Various different configurations exist, for example, the leg cuff 109 may comprise no elastic strands, 1 elastic strands, 2 elastic strands, 3 elastic strands, and so forth. The leg gather 108 may comprise no elastic strands, 1 elastic strand, 2 elastic strands, 3 elastic strands, and so forth. The elastic strands through the leg cuff 109 and the leg gather 108 may run parallel to the longitudinal axis or may run at a 30°-60° angle from the longitudinal axis. In one example, the elastic strands may run along lines of angular force through the leg gather 108 and/or the leg cuff 109. In one embodiment, the angle changes from parallel once an attachment point is made between the leg cuff and leg gather.

The length of the elastic strands may be the same or different within the leg cuff 109 and leg gather 108. For example, an elastic strand within the leg cuff 109 may be shorter than an elastic strand within the gather 108. The attachment of the elastic may also vary in the horizontal direction, such that one strand of elastic is closer to the center of the diaper, while another elastic may be closer to the leg side within the leg cuff 109 or leg gather 108.

The elastic strands in any of the embodiments may also be of varying decitex, depending on the desired elastic force. The spacing of multiple elastic strands can also vary, in some embodiments, depending on the desired elastic force.

FIG. 2 is an enlarged view of the right side crotch portion of the garment of FIG. 1. In this figure, the outer gather 108 comprises three strands of elastic 200, while the inner cuff 109 comprises none. FIG. 3 is an enlarged view of the left side embodiment of FIG. 1. In FIG. 3, the outer leg gather 108 comprises no elastic strands, while the inner cuff 109 comprises two strands of elastic 200. FIGS. 2 and 3 demonstrate that the combined cuff 110 bends differently depending on how the elastic strands are distributed.

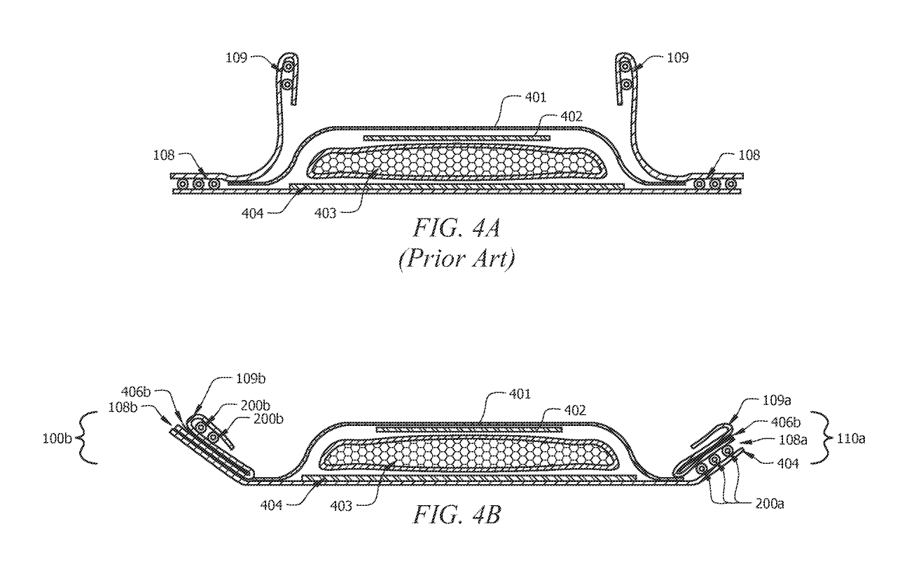

FIG. 4a illustrates a cross-section through the crotch section of a conventional diaper. This cross-section comprises from the top of the drawing to the bottom, a topsheet 401, an acquisition distribution layer (“ADL”) 402, an absorbent core 403, and the backsheet 404. Additionally, as depicted in FIG. 4a, diaper may also include a tissue or nonwoven cover 405 above, around, and/or below absorbent core 403. Cover 405 provides containment of the core materials preventing migration of fines and particulates during manufacturing and use and acts as a barrier preventing direct contact of the core and the wearer's skin. Cover 405 may also provide additional structural stability to the core. As shown in FIG. 4a, the conventional absorbent article has conventional double leg barrier construction comprising outer leg gather 108 and inner leg cuff 109.

FIG. 4b illustrates the cross section of two different embodiments of the combined leg cuff, cuff 110a and cuff 110b. The right side of the figure illustrates an embodiment of the combined leg cuff 110a where only the outer leg gather 108a comprises elastic strands 200a. The outer leg gather 108a and the inner leg cuff 109a are attached together through the use of an adhesive 406 which forms the combined cuff 110a. The left side of FIG. 4b illustrates an embodiment of the combined leg cuff 110b where only the inner leg cuff 109b comprises elastic strands 200b. The diaper may also use a cuff adhesive (not shown) to attach the inner leg cuff 109b to the topsheet 401 along the length of the diaper.

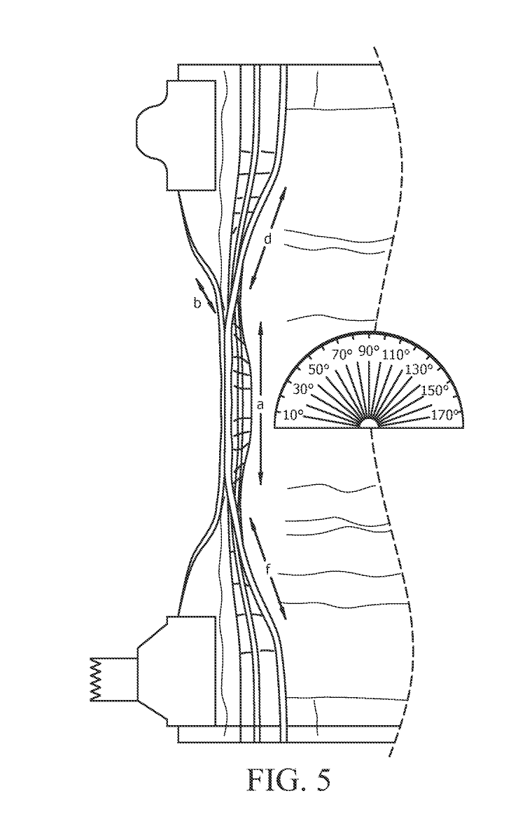

FIG. 5 illustrates an additional embodiment where the point of attachment between the leg cuff and leg gather is located towards the side panel and the forces involved in such attachment. The directions and forces are designated by letters in quotations. Referring to FIG. 5, typically in an absorbent article with a double leg barrier configuration, such as that described in in U.S. Pat. No. 4,695,278 to Lawson, and U.S. Pat. No. 4,704,116 to Enloe, there are three primary forces provided at least by certain elasticated regions: (1) the longitudinal force “a” along the length of the absorbent article provided by the elastication in the double leg barrier; (2) waist band force “b” provided by the elastication in the back waist region; (3) side panel force “c” provided by the elastication, if any, in side panel portion 104.

Referring to FIG. 5, an absorbent article with a preferred embodiment of a combined leg cuff of the present invention, such as combined leg cuff 110b, exhibits additional angular forces that can be adjusted and/or optimized to provide a desired fit around the wearer's leg. As shown, in addition to forces “a”, “b”, and “c” as described above, combined leg cuff 110 is positioned at a location near the front ear such that absorbent article 100 further preferably exhibits angular forces “d”, “e1”, “e2”, and “f”. Angular force “d” preferably runs in the diagonal direction between longitudinal force “a” and waist band force “b.” In one embodiment, angular force “d” has an angle between about 120-150 degrees, preferably about 120 degrees, with respect to the direction of longitudinal force “a”, which is considered to be about 90 degrees to the direction of waist band force “b”. Angular forces “e1” and “e2” are preferably positioned near back waist region 102. In a preferred embodiment, angular forces “e1” and “e2” have an angle between about 30-70 degrees respect to the side panel force “c”. Angular forces “e1” and “e2” can have the same angle or different angles, depending on the properties of combined leg cuff 110b, such as location, length, number of elastic strands. Angular force “f” preferably runs in the diagonal direction between longitudinal force “a” and front waist region 101. In one embodiment, angular force “d” has an angle between about 40-80 degrees, preferably about 60 degrees, with respect to the direction of longitudinal force “a”, which is considered to be about 90 degrees to the direction of waist band force “b”. In a preferred embodiment, the combination of these forces comes into play when the absorbent article is fitted on the body such that torque is developed by the angular forces created by combined leg cuff, e.g., 110b. This torque aids in creating a snug fit around the legs.

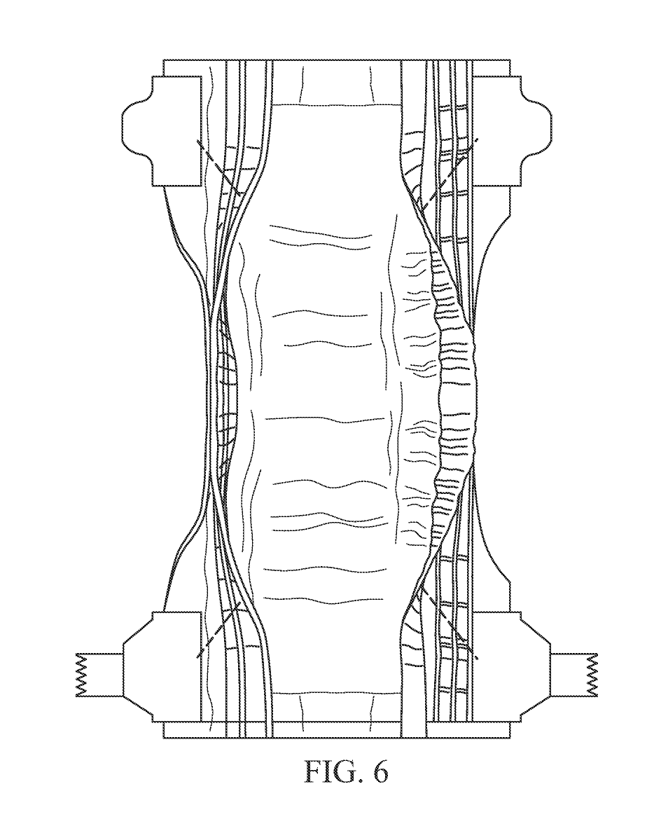

FIG. 6 illustrates an embodiment where combined leg cuff, e.g., 110b, is adapted to provide one or more additional elastics 120 attached at an angle with respect to side panel 104. As shown, a preferred embodiment also creates additional torque that aids in creating a snug fit around the legs of a wearer. The leg cuff or leg elastic would be allowed to snap back to some location, close to the attachment sites of the leg cuff or leg gather such that an additional band or bands would be placed as described.

In certain embodiments, the absorbent garment 100 also includes an acquisition distribution layer (ADL) 402. ADL 402 is generally a nonwoven sublayer placed between topsheet 401 and absorbent core 403. ADL 402 functions to acquire liquid and to distribute the liquid over a larger surface area of the absorbent core. The ADL 402 provides temporary liquid containment and facilitates the movement of fluid in the article, thereby allowing the article to approach its saturation capacity before failure. Absorbent core 403 in accordance with embodiments of the present application comprises unbleached pulp along with other absorbing materials. One suitable material for ADL 402 comprises a material having a basis weight of about 50 grams per square meter, and comprising a through-air-bonded-carded web of a homogeneous blend of 60% 3 denier bicomponent fiber comprising a polyester core/polyethylene sheath, commercially available from BASF Corporation, and 40% 6 denier polyester fibers, commercially available from Hoechst Celanese Corporation. Additionally, ADL 402 may comprise any of or combination of, and not limited to, spun, carded, meltblown, wetlaid, hydroentangled fibers or apertured film.

The various components of the diaper, e.g., topsheet 401, ADL 402, absorbent core 402, elastic portions, inner cuff 109, outer leg gather 108 and ear portions, etc., are integrally assembled together using various types of suitable attachment means that are well known in the art. These attachment means include the non-limiting examples of adhesives, sonic boding, thermal boding, or any combination thereof

The backsheet 404 is generally that portion of the diaper 100 positioned adjacent the garment facing surface of the absorbent core 403 which prevents the exudates absorbed and contained therein from soiling articles which may contact the diaper 100, such as bedsheets, clothes and undergarments. In preferred embodiments, the backsheet 404 is impervious to liquids (e.g., urine) and comprises a thin plastic film such as a thermoplastic film having a thickness of about 0.012 mm (0.5 mils) to about 0.051 mm (2.0 mils). Suitable backsheet films include those manufactured by Tredegar Industries Inc. of Terre Haute, Ind. and sold under the trade name X15306, X10962 and X10964. Other suitable backsheet materials may include breathable materials which permit vapors to escape from the diaper 100 while still preventing exudates from passing through the backsheet 404. Exemplary breathable materials may include materials such as woven webs, nonwoven webs, composite materials such as film-coated nonwoven webs, and macroporous films such as manufactured by Mitsui Toatsu Co. of Japan under the designation ESPOIR NO and by Tredegar Corp. of Richmond, Va. under the designation EXAIRE. Suitable breathable composite materials comprising polymer blends are available from Clopay Corporation, Cincinnati, Ohio under the name HYTREL blend P18-3097.

Backsheet 404 may be joined to topsheet 401, absorbent core 403 or any other element of the diaper 100 by any attachment means known in the art. For example, the attachment means may include a uniform continuous layer of adhesive, a patterned layer of adhesive, or an array of separate lines, spirals, or spots of adhesive. One preferred attachment means comprises an open pattern network of filaments of adhesive as disclosed in U.S. Pat. No. 4,573,986 issued Mar. 4, 1986 to Minetola et al. Other suitable attachment means include several lines of adhesive filaments which are swirled into a spiral pattern, as is illustrated by the apparatus and methods shown in U.S. Pat. No. 3,911,173 issued Oct. 7, 1975 to Sprague, Jr., U.S. Pat. No. 4,785,996 issued Nov. 22, 1978 to Ziecker, et al., and U.S. Pat. No. 4,842,666 issued Jun. 27, 1989 to Werenicz. Each of these patents are incorporated herein by reference. Adhesives which have been found to be satisfactory are manufactured by H. B. Fuller Company of St. Paul, Minn. and marketed as HL-1620 and HL-1358-XZP. Alternatively, the attachment means may comprise heat bonds, pressure bonds, ultrasonic bonds, dynamic mechanical bonds, or any other suitable attachment means or combinations of these attachment means as are known in the art.

Topsheet 401 is preferably positioned adjacent to the body of a user and between the user and absorbent core 403. Topsheet 401 may be joined to absorbent core 403 and/or to backsheet 404 by any attachment means suitable for such joining, such as discussed above. In some embodiments, topsheet 401 and backsheet 404 are joined directly in some locations and indirectly joined in other locations by directly joining topsheet 401 to other elements of diaper 100.

The topsheet 401 is preferably compliant, soft feeling, and non-irritating to the wearer's skin. Further, at least a portion of the topsheet 401 is liquid pervious, permitting liquids to readily penetrate through its thickness. A suitable topsheet 401 may be manufactured from a wide range of materials, such as porous foams, reticulated foams, apertured plastic films, or woven or nonwoven webs of natural fibers (e.g., wood or cotton fibers), synthetic fibers (e.g., polyester or polypropylene fibers), or a combination of natural and synthetic fibers. If the absorbent assemblies include fibers, the fibers may be spunbond, carded, wet-laid, meltblown, hydroentangled, or otherwise processed as is known in the art. For example, one suitable topsheet 201 comprising a web of staple length polypropylene fibers is manufactured by Veratec, Inc., a Division of International Paper Company, of Walpole, Mass. under the designation P-8.

Preferably, topsheet 401 is made of a hydrophobic material or is treated to be hydrophobic in order to isolate the wearer's skin from liquids contained in absorbent core 403. If topsheet 401 is made of a hydrophobic material, preferably at least the upper surface of topsheet 401 is treated to be hydrophilic so that liquids will transfer through the topsheet more rapidly. This diminishes the likelihood that body exudates will flow off of topsheet 401 rather than being drawn through the topsheet 401 and being absorbed by the absorbent core 403. Topsheet 401 can be rendered hydrophilic by treating it with a surfactant or by incorporating a surfactant into the topsheet. Suitable methods for treating topsheet 401 with a surfactant include spraying the topsheet 401 material with the surfactant and immersing the material into the surfactant. A more detailed discussion of such a treatment and hydrophilicity is contained in U.S. Pat. No. 4,988,344 issued Jan. 29, 1991 to Reising, et al. and U.S. Pat. No. 4,988,345 issued Jan. 29, 1991 to Reising. A more detailed discussion of some suitable methods for incorporating surfactant in the topsheet can be found in U.S. Statutory Invention Registration No. H1670, published on Jul. 1, 1997 in the names of Aziz et al. Each of these references is hereby incorporated by reference herein.

Any portion of the topsheet 401 may be coated with a lotion as is known in the art. The lotion may function alone or in combination with another agent as the hydrophobizing treatment described above. Topsheet 401 may also include or be treated with antibacterial agents. Further, the topsheet 401, the backsheet 404 or any portion of the topsheet 401 or backsheet 404 may be embossed and/or matte finished to provide a more cloth like appearance.

Absorbent core 403 may be comprised of a variety of different materials. For example, it may be constructed from materials such as hydrophilic fibers, cellulosic fluff such as wood pulp, superabsorbent hydrogel forming particles, synthetic polymeric, meltblown fibers, natural fibers, or combinations thereof. Example superabsorbent materials can be selected from natural, synthetic, and modified natural polymers and materials. The superabsorbent materials can be inorganic materials, such as silica gels, or organic compounds, such as crosslinked polymers. Suitable superabsorbent materials are available from various commercial vendors, such as BASF located in Charlotte, N.C., and Stockhausen GmbH & Co. KG, D-47805 Krefeld, Federal Republic of Germany. Additionally, superabsorbent polymers may be either biodegradable or non-biodegradable. Typically, a superabsorbent material is capable of absorbing at least about 15 times its weight in 0.9% saline, and desirably is capable of absorbing more than about 25 times its weight in 0.9% saline. In some embodiments, it may be preferred to utilize a superabsorbent material with a high pressure absorbency index (PAI) such as described in U.S. Pat. No. 5,601,542. Current implementations utilize materials that have an approximate 120 PAI rating. However, other embodiments may utilize a material having 30 PAI or higher.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A disposable absorbent garment comprising:
a front waist section;
a back waist section;
a crotch section disposed between the waist sections;
a liquid pervious topsheet;
a liquid impervious backsheet;
an absorbent core disposed between the topsheet and the backsheet;
a leg cuff attached to the topsheet at least in part parallel to a longitudinal axis at a first attachment region;
a leg gather section which runs along an outer edge region of the crotch section, wherein the leg gather section comprises an outer boundary and an inner boundary, wherein the outer boundary is defined by the edge of the crotch region and the inner boundary is defined by the first attachment region; and
wherein the leg gather region and leg cuff are attached together at least in part at a second attachment region.

2. The garment of claim 1, wherein the leg gather section comprises at least one of no elastic strands, one elastic strand, two elastic strands, three elastic strands, and more than three elastic strands.

3. The garment of claim 1, wherein the leg cuff comprises at least one of no elastic strands, one elastic strand, two elastic strands, three elastic strands, and more than three elastic strands.

4. The garment of claim 1, wherein the second attachment region is continuous along a line at least in part parallel to the longitudinal axis.

5. The garment of claim 1, wherein the second attachment region comprises a plurality of individual attachments.

6. The garment of claim 2, wherein the elastic strands at least in part are parallel to lines of angular force.

7. The garment of claim 1, where the second attachment region is made up of two or more individual attachment sites.

8. The garment of claim 1, wherein the second attachment region is made up of a single attachment site.

9. The garment of claim 8, wherein the single attachment site runs continuously along the edge of the crotch region.

10. The garment of claim 1, wherein the second attachment region is at least in part located in the front waist section, the back waist section, or both waist sections of the garment.

11. The garment of claim 1, wherein the second attachment region is located such that is closer to or inside of the back waist section and further from the back waist section.

12. The garment of claim 1, wherein the second attachment region is located toward the back waist section such that the angular force (d) changes from parallel (a) to about 40-80 degrees with respect to a waist band force (b) and 130-170 degrees (e1 and e2) with respect to the side panel force (c) while the point of attachment located toward the front ear is such that angular force (f) changes from parallel (a) to about 40-80 degrees with respect to an end of the second attachment region.

13. The garment of claim 1, wherein the leg gather region or the leg cuff comprises at least one elastic strand.

14. The garment of claim 1, wherein said leg cuff further comprises an elastic material configured to provide a force to hold the garment against a user when worn.

15. The garment of claim 1 wherein said leg gather further comprises an elastic material configured to provide a force to hold the garment against a user when worn.

16. The garment of claim 14 further comprising at least one additional elastic portion extending at an angle with respect to a side panel of said garment, said at least one additional elastic portion configured to provide an angular force to hold the garment against a user when worn.

17. The garment of claim 15 further comprising at least one additional elastic portion extending at an angle with respect to a side panel of said garment, said at least one additional elastic portion configured to provide an angular force to hold the garment against a user when worn. o
